(19) United States
(12) Patent Application Publication
(10) Pub. No.: US 2026/0345708 A1
(43) Pub. Date: Nov. 5, 2026

(54) COMPOSITION FOR PREVENTING OR TREATING RADIATION-RESISTANT HEAD AND NECK CANCER, COMPRISING MELANDRIUM HERB EXTRACT AS ACTIVE INGREDIENT

(71) Applicant: Bonglee KIM

(72) Inventor: Bonglee KIM

(21) Appl. No.: 19/124,577

(22) PCT Filed: Oct. 24, 2023
(86) PCT No.: PCT/KR2023/016562

§ 371 Date: Apr. 25, 2025

(51) Int. Cl. A61K 36/36; A61P 35/00

(52) U.S. Cl. CPC A61K 36/36; A61P 35/00; A61K 2236/333; A61K 2236/53

(57) ABSTRACT

The present invention relates to a composition for preventing or treating radiation-resistant head and neck cancer, the composition comprising a melandrium herb extract as an active ingredient. The melandrium herb extract according to the present invention was found to effectively kill radiation-resistant head and neck cancer cell lines. In addition, the melandrium herb extract was found to induce apoptosis of radiation-resistant head and neck cancer cell lines by inducing the production of ROS related to apoptosis. In addition, it was discovered that the melandrium herb extract could more effectively kill radiation-resistant head and neck cancer cell lines when used in combination with radiation treatment than when used by itself.

### TECHNICAL FIELD

The present disclosure relates to a composition for preventing or treating radiation-resistant head and neck cancer, comprising a melandrium herb extract as an active ingredient.

### BACKGROUND ART

Cancer has a high death rate worldwide, and is the most common death cause next to cardiovascular disease in the Western society. In particular, lung cancer is increasing due to the aging of population, an increase in the smoking population, and air pollution. In addition, due to the westernization of diets, colon cancer, breast cancer, prostate cancer, and the like tend to continuously increase by the generalized consumption of high-fat foods, a rapid increase in environmental pollutants, and an increase in alcohol consumption. In this situation, the creation of anticancer materials has been urgently required by enabling early prevention and treatment of cancer to contribute to enhancement of human health, improvement of the quality of health life, and promotion of human health care. In order to overcome cancer, there is a strong need for innovative cancer research focusing on a deeper understanding of the fundamental mechanisms of DNA damage/repair, apoptosis, tumorigenesis, and treatment in molecular aspects, which may ultimately lead to a breakthrough in cancer therapy.

Head and neck cancer is known to be the sixth most common cancer among entire cancer worldwide, with approximately 600,000 patients each year. The head and neck area refers to an area between the lower brain and the upper chest, and the head and neck area contains many organs that function in eating and producing speech, such as the nasal cavity, tongue, mouth, larynx, and salivary glands. Cancer occurring in these organs is collectively referred to as head and neck cancer. Genetic factors, smoking, and drinking are known to cause head and neck cancer. Head and neck cancer has many limitations in treatment compared to cancers of other organs because of specificity of the face and neck, and is also a place which is fatal when it progresses and often causes functional and external disorders after treatment. The pathological origins of occurring diseases are diverse, and even diseases with the same pathology have the characteristic of showing completely different prognoses depending on a location of occurrence. As the population ages and drinking and smoking increase, the incidence of head and neck cancer is also on the rise, accounting for 4 to 5% of all cancers. Statistically, the head and neck cancer is a relatively common cancer, ranking seventh based on cancer occurrence by organ and fifth based on male patients. Specifically, the types of head and neck cancer broadly include sinonasal cavity cancer, oral cancer, nasopharyngeal cancer, oropharyngeal cancer, hypopharyngeal cancer, laryngeal cancer, cervical esophageal cancer, salivary gland cancer, salivary tumor, malignant lymphoma, malignant melanoma, and various soft tissue cancers. Another important feature of head and neck cancer is that secondary cancers frequently develop in other organs. Depending on a report, it is known that about 10 to 40% of secondary cancer occurs in areas other than the head and neck, such as the lungs, stomach, or esophagus, throughout the lifetime. As such, the reason why the incidence of secondary cancer is high is that head and neck cancer, lung cancer, stomach cancer, and esophageal cancer share carcinogenic factors. A treatment method for head and neck cancer is mainly used with surgery, radiation therapy, and chemotherapy.

Meanwhile, cells within the body are eliminated through an apoptosis process. However, cancer cells proliferate indefinitely through continuous division. To eliminate these cancer cells, the death needs to be induced. PARP plays a role in repairing DNA when damaged. The PARP may not function when apoptosis occurs. Therefore, the amount of PARP appears to decrease in cells undergoing apoptosis. Reactive oxygen species (ROS) is one of factors that cause apoptosis in cancer cells. Staining with DCF-DA may determine the degree of ROS generation, and an increase in ROS may be confirmed by increased cancer cell death.

Meanwhile, the melandrium herb refers to the aerial part of the ripe fruit of Melandrium firmum Rohrbach, and is also called Bulryuhaeng and Wangbulryu in Korean. The melandrium herb promotes blood circulation, reduces swelling, and stops bleeding. In addition, it has been reported in pharmacological experiments to have an effect of enhancing immune functions, and the melandrium herb is a medicinal herb used for boil, malignant furuncle, nosebleed, traumatic bleeding, joint pain, gonorrhea, and the like.

Accordingly, the present inventors confirmed that the melandrium herb extract induced apoptosis of radiation-resistant head and neck cancer, and then completed the present disclosure.

### DISCLOSURE

### Technical Problem

An object of the present disclosure is to provide a pharmaceutical composition for preventing or treating head and neck cancer including a melandrium herb (Melandrium firmum Rohrbach) extract as an active ingredient.

Another object of the present disclosure is to provide an anticancer adjuvant agent for head and neck cancer including a melandrium herb (Melandrium firmum Rohrbach) extract as an active ingredient.

Yet another object of the present disclosure is to provide a food composition for preventing or alleviating head and neck cancer including a melandrium herb (Melandrium firmum Rohrbach) extract as an active ingredient.

Still another object of the present disclosure is to provide a pharmaceutical composition for preventing or treating radiation-resistant head and neck cancer including a melandrium herb (Melandrium firmum Rohrbach) extract as an active ingredient.

Still another object of the present disclosure is to provide an anticancer adjuvant agent for radiation-resistant head and neck cancer including a melandrium herb (Melandrium firmum Rohrbach) extract as an active ingredient.

Still another object of the present disclosure is to provide a food composition for preventing or alleviating radiation-resistant head and neck cancer including a melandrium herb (Melandrium firmum Rohrbach) extract as an active ingredient.

Still another object of the present disclosure is to provide a method for inhibiting the proliferation of a head and neck cancer cell line, including treating a head and neck cancer cell line with a melandrium herb (Melandrium firmum Rohrbach) extract in vitro.

Still another object of the present disclosure is to provide a method for treating head and neck cancer including administering to a subject a melandrium herb (Melandrium firmum Rohrbach) extract in a pharmaceutically effective amount.

### Technical Solution

In order to achieve the object, an aspect of the present disclosure provides a pharmaceutical composition for preventing or treating head and neck cancer including a melandrium herb (Melandrium firmum Rohrbach) extract as an active ingredient.

Further, another aspect of the present disclosure provides an anticancer adjuvant agent for head and neck cancer including a melandrium herb (Melandrium firmum Rohrbach) extract as an active ingredient.

Further, yet another aspect of the present disclosure provides a food composition for preventing or alleviating head and neck cancer including a melandrium herb (Melandrium firmum Rohrbach) extract as an active ingredient.

Further, still another aspect of the present disclosure provides a pharmaceutical composition for preventing or treating radiation-resistant head and neck cancer including a melandrium herb (Melandrium firmum Rohrbach) extract as an active ingredient.

Further, still another aspect of the present disclosure provides an anticancer adjuvant agent for radiation-resistant head and neck cancer including a melandrium herb (Melandrium firmum Rohrbach) extract as an active ingredient.

Further, still another aspect of the present disclosure provides a food composition for preventing or alleviating radiation-resistant head and neck cancer including a melandrium herb (Melandrium firmum Rohrbach) extract as an active ingredient.

Further, still another aspect of the present disclosure provides a method for inhibiting the proliferation of a head and neck cancer cell line, including treating a head and neck cancer cell line with a melandrium herb (Melandrium firmum Rohrbach) extract in vitro.

Further, still another aspect of the present disclosure provides a method for treating head and neck cancer including administering to a subject a melandrium herb (Melandrium firmum Rohrbach) extract in a pharmaceutically effective amount.

### Advantageous Effects

The present disclosure relates to a composition for preventing or treating head and neck cancer, the composition comprising a melandrium herb extract as an active ingredient. The melandrium herb extract according to the present disclosure was found to effectively kill radiation-resistant head and neck cancer cell lines. In addition, the melandrium herb extract was found to induce apoptosis of head and neck cancer cell lines by inducing the production of ROS related to apoptosis. In addition, the melandrium herb extract was found to more effectively kill radiation-resistant head and neck cancer cell lines when used in combination with radiation treatment than when treated alone, and thus can be usefully used for the treatment of radiation-resistant head and neck cancer.

### DESCRIPTION OF DRAWINGS

FIG. 1 is a diagram confirming cytotoxicity in radiation-resistant head and neck cancer according to treatment with a melandrium herb extract of the present disclosure.

FIG. 2 is a diagram confirming induction of ROS production in radiation-resistant head and neck cancer cell lines according to treatment with a melandrium herb extract of the present disclosure.

FIG. 3 is a diagram confirming apoptosis in radiation-resistant head and neck cancer cell lines according to combined treatment with a melandrium herb extract of the present disclosure and radiation.

### BEST MODE

Hereinafter, examples of the present disclosure will be described in detail with reference to the accompanying drawings. In the following description, detailed descriptions of techniques well-known to those skilled in the art may be omitted. Further, in describing the present disclosure, the detailed description of associated known functions or constitutions will be omitted if it is determined to unnecessarily make the gist of the present disclosure unclear. In addition, terminologies used in the present disclosure are terminologies used to properly express preferred examples of the present disclosure, which may vary according to a user, an operator's intention, or customs in the art to which the present disclosure pertains.

Accordingly, definitions of the terminologies need to be described based on contents throughout this specification. Throughout this specification, unless explicitly described to the contrary, when a certain part “comprises” a certain component, it will be understood to imply the inclusion of stated elements, not the exclusion of any other elements.

The present disclosure provides a pharmaceutical composition for preventing or treating head and neck cancer including a melandrium herb (Melandrium firmum Rohrbach) extract as an active ingredient.

As used in the present disclosure, the term “prevention” refers to any action that suppresses the symptoms of a specific disease or delays its progression by administering the composition of the present disclosure.

As used in the present disclosure, the term “treatment” refers to any action that improves or beneficially changes the symptoms of a specific disease by administering the composition of the present disclosure.

The pharmaceutical composition of the present disclosure may further include an adjuvant in addition to the active ingredient. The adjuvant may be used with any adjuvant known in the art without limitation, but further include, for example, a Freund's complete adjuvant or an incomplete adjuvant to increase the effect thereof.

The pharmaceutical composition according to the present disclosure may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present disclosure may be formulated and used in the form of oral formulations, such as powders, granules, tablets, capsules, suspensions, emulsions, syrups, aerosols, etc., external preparations, suppositories, or sterile injectable solutions according to each conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one or more excipients, for example, starch, calcium carbonate, sucrose, lactose, gelatin, etc. with the active ingredient. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used diluents, such as water and liquid paraffin. Formulations for parenteral administration include sterile aqueous solutions, non-aqueous solvents, suspensions, emulsions, lyophilized agents, and suppositories. As the non-aqueous solvent and the suspension, propylene glycol, polyethylene glycol, vegetable oil such as olive oil, injectable ester such as ethyl oleate, etc. may be used. As the base material of the suppository, witepsol, Tween 61, cacao butter, laurinum, glycerogelatin, etc. may be used.

The pharmaceutical composition according to the present disclosure may be administered to a subject through various routes. All methods of administration may be expected, and the pharmaceutical composition may be administered by, for example, oral, intravenous, intramuscular, subcutaneous, and intraperitoneal injection.

The dose of the pharmaceutical composition according to the present disclosure is selected in consideration of the age, body weight, sex, physical conditions, and the like of a subject. It is obvious that the concentration of the active ingredient included in the pharmaceutical composition may be variously selected according to a subject, and preferably included in the pharmaceutical composition at a concentration of 0.01 to 5,000 μg/ml. When the concentration is less than 0.01 μg/ml, pharmaceutical activity may not be exhibited, and when the concentration is more than 5,000 μg/ml, toxicity to the human body may be exhibited.

According to one embodiment of the present disclosure, the melandrium herb extract may be extracted with a solvent selected from the group consisting of water, lower alcohols having C1 to C4, a lower alcohol aqueous solution, ethyl acetate, hexane, and chloroform, preferably a lower alcohol aqueous solution, but is not limited thereto.

According to one embodiment of the present disclosure, the head and neck cancer may further include radiation-resistant head and neck cancer.

The “radiation-resistant” of the present disclosure refers to the degree to which a tumor may be cured in a high unit in radiation, which is called a tumor curable dose, and means the state of a tumor with a high radiocurable dose.

According to one embodiment of the present disclosure, the head and neck cancer may be selected from the group consisting of oral cancer, oropharyngeal cancer, hypopharyngeal cancer, laryngeal cancer, salivary gland cancer, nasal cancer, paranasal sinus cancer, nasopharyngeal cancer, cervical esophageal cancer, salivary gland cancer, malignant lymphoma, malignant melanoma, and soft tissue cancer, and preferably oral cancer, but is not limited thereto.

According to one embodiment of the present disclosure, the melandrium herb extract may induce apoptosis of head and neck cancer, and the inducing of the apoptosis may be inducing the production of reactive oxygen species (ROS).

According to one embodiment of the present disclosure, the melandrium herb extract may be treated in combination with radiation.

Further, the present disclosure provides an anticancer adjuvant agent for head and neck cancer including a melandrium herb (Melandrium firmum Rohrbach) extract as an active ingredient.

The “anticancer adjuvant” of the present invention is an agent that may improve, enhance or increase an anticancer effect of an anticancer agent, and does not show anticancer activity by itself, but may be an agent capable of improving, enhancing or increasing the anticancer effect of the anticancer agent when used together with the anticancer agent. In addition, when an agent exhibiting a concentration-dependent anticancer activity is used together with the anticancer agent at a level that does not exhibit anticancer activity by itself, the anticancer adjuvant may be an agent capable of improving, enhancing or increasing the anticancer effect of the anticancer agent.

The anticancer adjuvant may be administered through any general route as long as the anticancer adjuvant may reach a target tissue. The anticancer adjuvant of the present invention may be administered intraperitoneally, intravenously, intramuscularly, subcutaneously, intradermally, orally, intranasally, pulmonaryly, or intrarectally according to a desired purpose, but is not limited thereto. In addition, the anticancer adjuvant may be administered by any device capable of transferring an active substance to a target cell.

The present disclosure provides a food composition for preventing or alleviating head and neck cancer including a melandrium herb (Melandrium firmum Rohrbach) extract as an active ingredient.

As used in the present disclosure, the “improvement” means all actions that at least reduce parameters associated with conditions to be treated, e.g., the severity of symptoms.

In addition to containing the active ingredient of the present disclosure, the food composition of the present disclosure may contain various flavoring agents, natural carbohydrates, or the like as an additional ingredient, like conventional food compositions.

Examples of the above-described natural carbohydrates include conventional sugars, including monosaccharides, such as glucose, fructose, etc.; disaccharides, such as maltose, sucrose, etc.; and polysaccharides, such as dextrin, cyclodextrin, etc., and sugar alcohols such as xylitol, sorbitol, erythritol, etc. The above-described flavoring agents may be advantageously used with natural flavoring agents (thaumatin), stevia extracts (e.g., rebaudioside A, glycyrrhizin, etc.), and synthetic flavoring agents (saccharin, aspartame, etc.). The food composition of the present disclosure may be formulated in the same manner as the pharmaceutical composition to be used as a functional food or added to various foods. The foods capable of adding the composition of the present disclosure include, for example, beverages, meat, chocolate, foods, confectionery, pizza, ramen, other noodles, gums, candies, ice creams, alcohol beverages, vitamin complexes, health food supplements, etc.

In addition, the food composition may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, and the like, in addition to the extract as the active ingredient. In addition, the food composition of the present disclosure may contain pulps for preparing natural fruit juice, fruit juice beverages, and vegetable beverages.

The functional food composition of the present disclosure may be prepared and processed in the form of tablets, capsules, powders, granules, liquids, pills, etc., for the purpose of prevention or treatment of head and neck cancer. In the present disclosure, the ‘health functional food composition’ refers to foods prepared and processed by using raw materials or ingredients with functionality, which are useful for the human body according to the Art No. 6727 on Health Functional Food, and means foods taken for adjusting nutrients for the structures and functions of the human body or obtaining a useful effect on health applications such as physiological actions. The health functional food of the present disclosure may include conventional food additives, and the suitability as the food additives is determined by the specifications and standards for the corresponding item in accordance with the general rules of the Food Additives Codex, general test methods, etc., that are approved by the Food and Drug Administration, unless otherwise specified. The items disclosed in the ‘Food Additives Codex’ may include, for example, chemical composites such as ketones, glycine, calcium citrate, nicotinic acid, cinnamic acid, etc.; natural additives such as persimmon color, licorice extract, crystal cellulose, Kaoliang color, guar gum, etc.; mixed formulations such as sodium L-glutamic acid formulations, noodle additive alkali agents, preservative formulations, tar color formulations, etc. For example, the healthy functional food in the form of tablets may be prepared by granulating a mixture obtained by mixing the active ingredient of the present disclosure with an excipient, a binder, a disintegrant, and other additives by conventional known methods, and then compression-molding the mixture by adding a slip modifier and the like, or directly compression-molding the mixture. In addition, the healthy functional food in the form of tablets may also contain a flavor enhancer or the like as needed. In the health functional food in the form of capsules, hard capsules may be prepared by filling a mixture mixed with the active ingredient of the present disclosure and additives such as excipients into conventional hard capsules, and soft capsules may be prepared by filling a mixture mixed with the active ingredient of the present disclosure and additives such as excipients into capsule bases such as gelatin. The soft capsules may contain a plasticizer such as glycerin or sorbitol, a colorant, a preservative, and the like, if necessary. The healthy functional food in the form of pills may be prepared by molding a mixture obtained by mixing the active ingredient of the present disclosure with an excipient, a binder, a disintegrant, etc. by conventional known methods, and may also be coated with white sugar or other coating agents or surface-coated with materials such as starch and talc, if necessary. The healthy functional food in the form of granules may be prepared by granulizing a mixture obtained by mixing the active ingredient of the present disclosure with an excipient, a binder, a disintegrant, etc. by conventional known methods and may contain a flavoring agent, a flavor enhancer, etc., if necessary.

Further, the present disclosure provides a pharmaceutical composition for preventing or treating radiation-resistant head and neck cancer including a melandrium herb (Melandrium firmum Rohrbach) extract as an active ingredient.

Further, the present disclosure provides an anticancer adjuvant agent for radiation-resistant head and neck cancer including a melandrium herb (Melandrium firmum Rohrbach) extract as an active ingredient.

Further, the present disclosure provides a food composition for preventing or alleviating radiation-resistant head and neck cancer including a melandrium herb (Melandrium firmum Rohrbach) extract as an active ingredient.

Further, the present disclosure provides a method for inhibiting the proliferation of a head and neck cancer cell line, including treating a head and neck cancer cell line with a melandrium herb (Melandrium firmum Rohrbach) extract in vitro.

Further, the present disclosure provides a method for treating head and neck cancer including administering to a subject a melandrium herb (Melandrium firmum Rohrbach) extract in a pharmaceutically effective amount.

The treatment method of the present disclosure includes administering the melandrium herb (Melandrium firmum Rohrbach) extract to a subject in a therapeutically effective amount. It is preferred that a specific therapeutically effective amount for a specific subject is differently applied depending on various factors including the type and degree of a response to be achieved, a specific composition including whether other agents are used in some cases, the age, body weight, general health conditions, sex, and diet of a subject, an administration time, an administration route, a secretion rate of the composition, a duration of treatment, and a drug used in combination or simultaneously with the specific composition, and similar factors well known in the medical field. A daily dose may be 0.0001 to 100 mg/kg, preferably 0.01 to 100 mg/kg, based on the amount of the pharmaceutical composition of the present disclosure, and may be administered 1 to 6 times a day. However, it is obvious to those skilled in the art that the dosage or dose of each active ingredient does not cause side effects by including the content of each active ingredient too high. Therefore, the effective amount of the composition suitable for the purpose of the present disclosure is preferably determined in consideration of the aforementioned matters.

The subject is applicable to any mammal, and the mammal includes not only humans and primates, but also livestock such as cattle, pig, sheep, horse, dog, and cat.

The melandrium herb (Melandrium firmum Rohrbach) extract of the present disclosure may be administered to mammals such as mice, rats, livestock, and humans through various routes. All methods of administration may be expected, and for example, the pharmaceutical composition may be administered by oral, rectal or intravenous, intramuscular, subcutaneous, intrauterine dural or intracerebroventricular injection.

### Modes

Hereinafter, the present disclosure will be described in more detail through Examples. These Examples are just to explain the present disclosure in more detail, and it will be apparent to those skilled in the art that the scope of the present disclosure is not limited to these Examples.

### <Preparation Example 1> Preparation of Melandrium Herb Extract

In order to confirm an anticancer effect of a melandrium herb (Melandrium firmum Rohrbach) extract of the present disclosure on head and neck cancer, the melandrium herb extract was prepared. Specifically, a melandrium herb grown in Hongcheon, Gangwon-do was mixed with 25% ethanol (a mixture of 25 v/v % ethanol and 75 v/v % distilled water) and extracted for 48 hours. Thereafter, the extract was filtered and concentrated under reduced pressure to obtain a 25% ethanol extract.

### <Example 1> Confirmation of Cytotoxicity of Melandrium Herb Extract on Radiation-Resistant Head and Neck Cancer Cell Line

The cytotoxicity of the melandrium herb extract of the present disclosure on radiation-resistant head and neck cancer was confirmed. Specifically, a radiation-resistant head and neck cancer cell line Moc1-RR (Kyung Hee Medical Center, Seoul, Korea) was used, and the culture medium was used with a medium prepared by filtering an IMDM medium containing 31.3% nutrient mixture, 5% inactivated fetal bovine serum, and 1% penicillin-streptomycin, and then adding 5 mg/mL Insulin, Hydrocortisone, and EGF. 100 μl of the Moc1-RR cell line was dispensed into a 96-well plate to be a concentration of 1.0×10⁴ cells and then cultured in a 5% CO₂ incubator (MCO-15AC, Sanyo, Osaka, Japan) at 37 °C. After removing the medium, the cultured cells were treated with 100 μl of the melandrium herb extract of Preparation Example 1 at concentrations of 0, 3.125, 6.25, 12.5, 25 and 50 μg/ml, respectively, and then further cultured for 24 hours. Thereafter, the cultured cells were treated with 10 μl of an EZ-Cytox reagent (DoGen) and reacted for 1 hour. After the reaction was completed, the cell viability was confirmed by measuring the absorbance at a wavelength of 450 nm using a microplate reader (BKMPR-1101).

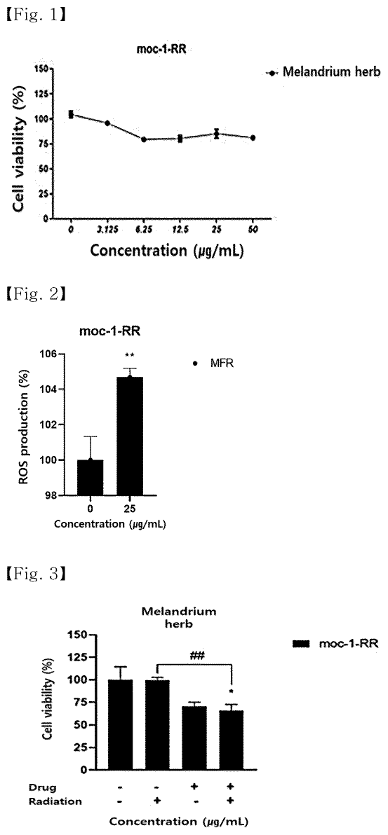

As a result, as shown in FIG. 1, the melandrium herb extract of the present disclosure was confirmed to kill the radiation-resistant head and neck cancer cell line Moc1-RR in a concentration-dependent manner, thereby confirming cytotoxicity on cancer cell lines.

### <Example 2> Confirmation of ROS Production of Melandrium Herb Extract in Radiation-Resistant Head and Neck Cancer Cell Line

It was confirmed whether the melandrium herb extract of the present disclosure promoted the production of ROS that induced apoptosis in a radiation-resistant head and neck cancer cell lines. Specifically, DCF-DA assay was performed to confirm the production of ROS, and 100 μl of a Moc1-RR cell line was dispensed into a 96-well plate to be a concentration of 1.0×10⁴ cells, and cultured in a 5% CO₂ incubator at 37 °C. The cultured cells were treated with 20 μM DCF-DA after removing the medium and reacted for 45 minutes. After the reaction was completed, the medium was removed and the cells were washed with an IMDM medium without phenol red. Thereafter, the cells were treated with 100 μl of the melandrium herb extract at concentrations of 0 and 25 μg/ml, and then further cultured for 4 hours. After the culture was completed, ROS production was confirmed by observing fluorescence at 485 nm and 535 nm using a microplate reader.

As a result, as shown in FIG. 2, the melandrium herb extract induced the production of ROS in the radiation-resistant head and neck cancer cell line, thereby confirming the induction of the death of the radiation-resistant head and neck cancer cell line.

### <Example 3> Confirmation of Combined Effect of Melandrium Herb Extract and Radiation

It was confirmed whether the melandrium herb extract of the present disclosure killed radiation-resistant head and neck cancer in combination with radiation treatment. Specifically, 100 μl of a Moc1-RR cell line was dispensed into two 96-well plates to be a concentration of 5×10⁴ cells, respectively, and then cultured in a 5% CO₂ incubator at 37 °C. After the culture was completed, the medium was removed, and the plates were treated with 100 μl of the melandrium herb extract at concentrations of 0 and 25 μg/ml, respectively, and then cultured for 24 hours. After the culture was completed, the medium was replaced with a new IMDM medium, and one plate was treated with 4 Gy of radiation. Then, both of the plates were further cultured for 48 hours. After the culture was completed, cell viability was confirmed using the same method as in Example 1.

As a result, as shown in FIG. 3, it was confirmed that when the Moc1-RR cell line was treated with the melandrium herb extract of the present disclosure in combination with radiation, an effect of inducing apoptosis was significantly increased compared to treatment with the melandrium herb extract alone, thereby confirming an effect of combined treatment with radiation.

Therefore, it was confirmed that the melandrium herb extract of the present disclosure effectively killed radiation-resistant head and neck cancer cell lines. In addition, the melandrium herb extract was found to induce apoptosis of radiation-resistant head and neck cancer cell lines by inducing the production of ROS related to apoptosis. In addition, it was confirmed that the melandrium herb extract could more effectively kill radiation-resistant head and neck cancer cell lines when used in combination with radiation treatment than when treated with the melandrium herb extract alone.

1. A method for treating head and neck cancer comprising administering to a subject in need thereof a melandrium herb (Melandrium firmum Rohrbach) extract in a pharmaceutically effective amount.

2. The method of claim 1, wherein the melandrium herb extract is extracted with a solvent selected from the group consisting of water, lower alcohols of C1 to C4, a lower alcohol aqueous solution, ethyl acetate, hexane, and chloroform.

3. The method of claim 1, wherein the head and neck cancer further includes radiation-resistant head and neck cancer.

4. The method of claim 1, wherein the head and neck cancer is selected from the group consisting of oral cancer, oropharyngeal cancer, hypopharyngeal cancer, laryngeal cancer, salivary gland cancer, nasal cancer, paranasal sinus cancer, nasopharyngeal cancer, cervical esophageal cancer, salivary gland cancer, malignant lymphoma, malignant melanoma, and soft tissue cancer.

5. The method of claim 1, wherein the melandrium herb extract induces apoptosis of head and neck cancer.

6. The method of claim 5, wherein the inducing of the apoptosis is inducing the production of reactive oxygen species (ROS).

7. The method of claim 1, wherein the melandrium herb extract is treated in combination with radiation.

8. A method for treating radiation-resistant head and neck cancer comprising administering to a subject in need thereof a melandrium herb (Melandrium firmum Rohrbach) extract in a pharmaceutically effective amount.

9. A method for inhibiting the proliferation of a head and neck cancer cell line, comprising treating a head and neck cancer cell line with a melandrium herb (Melandrium firmum Rohrbach) extract in vitro.

10.-14. (canceled)
